(19) United States
(12) Patent Application Publication
(10) Pub. No.: US 2026/0085247 A1
(43) Pub. Date: Mar. 26, 2026

(54) METHOD FOR PRODUCING C2-C4 OLEFINS FROM METHANOL AND ETHANOL

(71) Applicant: BASF SE, Ludwigshafen am Rhein (DE)

(72) Inventors: Andre BADER, Ludwigshafen am Rhein (DE); Lee Russell COLLINS, Ludwigshafen am Rhein (DE); Andreas JOERKE, Ludwigshafen am Rhein (DE); Paul-Vinzent STROBEL, Ludwigshafen am Rhein (DE); Robert Peter Michael FRANZ, Ludwigshafen am Rhein (DE)

(21) Appl. No.: 19/110,197

(22) PCT Filed: Sep. 4, 2023
(86) PCT No.: PCT/EP2023/074185

§ 371 Date: Mar. 10, 2025

(30) Foreign Application Priority Data
Sep. 12, 2022 (EP) 22195156.9
Oct. 25, 2022 (EP) 22203569.3

(51) Int. Cl. C10G 3/00; C10G 49/00

(52) U.S. Cl. CPC C10G 3/42; C10G 3/54; C10G 49/002; C10G 2400/20

(57) ABSTRACT

The invention relates to a method for producing C2-C4 olefins from methanol and ethanol, said method having the steps of: A) feeding a methanol- and optionally ethanol-containing feed flow A into a dimethyl ether fixed-bed reactor and catalytically reacting methanol to form dimethyl ether, wherein a product flow A1 containing dimethyl ether, methanol, ethanol and steam is obtained; B) mixing the flow A1 with at least one hydrocarbon return flow R containing C2-C6 hydrocarbons and catalytically reacting the mixture in an olefin fixed-bed reactor to form a raw product flow B containing C2-C4 olefins, C5-C6 hydrocarbon and C7+ hydrocarbons; C) cooling the raw product flow B, wherein a hydrocarbon raw product flow C is obtained; D) separating the hydrocarbon raw product flow C in a propylene-containing value product flow, optionally an ethylene-containing value product flow, a butene-containing value product flow, at least one C5-C6 hydrocarbon-containing return flow and at least one C6+ hydrocarbon-containing auxiliary product flow; E) returning a part of the C2-C4 olefins and at least a part of the C5-C6 hydrocarbons as one or more hydrocarbon return flows in step B); F) recovering a propylene-containing value product flow, an ethylene-containing value product flow and optionally a butene-containing value product flow; G) discharging the C6+ hydrocarbon-containing auxiliary product flow; characterised in that the flow A, in relation to methanol and ethanol, contains <1 wt. % or 30 to 50 wt. % ethanol, wherein, in relation to 100 wt. % of the C2-C4 olefins recovered as value products, 30 to 60 wt. % ethylene, 30 to 60 wt. % propylene, and 0 to 30 wt. % butene are recovered as value products, and, in relation to the C2-C4 olefins contained in the raw product flow B, 0 to 40% of the ethylene, 40 to 90% of the propylene, and 0 to 100% of the butene are fed back in step B), or the flow A, in relation to methanol and ethanol, contains 1 to 30 wt. % ethanol, wherein, in relation to 100 wt. % of the C2-C4 olefins recovered as value products, 0 to 20 wt. % ethylene, 70 to 100 wt. % propylene, and 1 to 20 wt. % butene are recovered as value products, and, in relation to the 40 C2-C4 olefins contained in the raw product flow B, 0 to 100% of the ethylene, 0 to 20% of the propylene and 40 to 100% of the butene are fed back in step B).

The invention relates to a process for preparing C₂-C₄ olefins from methanol, with or without ethanol.

It is known that propylene can be produced by converting a methanol/dimethyl ether mixture in a fixed bed reactor (methanol-to-propylene, MTP reactor). Known fixed bed reactors are operated with zeolite catalysts at temperatures of about 480° C.

US 2010/145125 A1 discloses a process for preparing light olefins by the conversion of methanol and ethanol. The process comprises the feeding of a first part-feed via a distributor at the base of a fluidized bed reactor to a reaction zone comprising a catalyst, the feeding of a second part-feed from at least one point above the distributor to the reaction zone, the contacting of the feed with the catalyst and reacting to give a stream comprising ethylene and propylene, where the first and second part-feeds each independently comprise methanol and/or ethanol, with the proviso that the feed overall comprises both methanol and ethanol, and the weight ratio of methanol to ethanol in the feed overall is in the range from 99:1 to 0.1:1.

CN 216106699 U discloses a process for preparing ethylene, propylene and butenes by catalytic dehydration of methanol. The system comprises a reaction unit and a separation unit, wherein the reaction unit comprises a preliminary reactor, a process vapor column and a main reactor, and the separation unit comprises a quencher and a compressor. The main reactor comprises a ZSM-5 molecular sieve catalyst; the separating unit comprises an ethylene column, a propylene column and a butenes column. Ethane, propane, butane and C₅ and C₆ hydrocarbons are partly recycled into the main reactor; further components are discharged as by-products. The unit can also be used to prepare butenes while the amount of recycled hydrocarbons is reduced and the energy consumption of the unit is lowered. Only saturated hydrocarbons are recycled into the main reactor.

CN 110218138 A discloses a process for increasing the yield of olefin in a methanol-to-propylene process (MTP process), in which C₂ hydrocarbons, C₄ hydrocarbons and C₅-C₇ hydrocarbons are recycled into the MTP reactor. In one example, in a 500 000 t/a methanol-to-propylene plant, the methanol feed is 210 t/h, and those of the recycled C₂, C₄ and C₅-C₇ hydrocarbons are 27 t/h, 40 t/h and 150 t/h respectively. Ethylene production is 1.5 t/h; propylene production is 60 t/h. There is no mention of co-feeding of ethanol. The product of value obtained is very predominantly propylene.

It is an object of the invention to provide a flexible process for preparing C₂-C₄ olefins from methanol in a methanol-to-olefin process (MTO process), in which the proportions of ethylene, propylene and butenes in the product of value streams obtained from the process can be varied within wide ranges, and the volume of the recycle streams recycled into the MTO process can be reduced overall.

The object is achieved by a process for preparing C₂-C₄ olefins from methanol, with or without ethanol, comprising the steps of:
A) feeding a feed stream A comprising methanol, with or without ethanol, into a dimethyl ether fixed bed reactor and catalytically converting methanol to dimethyl ether to obtain a product stream A1 comprising dimethyl ether, methanol and water vapor, with or without ethanol and ethylene;
B) mixing stream A1 with at least one hydrocarbon recycle stream R comprising C₂-C₆ hydrocarbons and catalytically converting it in an olefin fixed bed reactor to a crude product stream B comprising C₂-C₄ olefins, C₅-C₆ hydrocarbon and C₇⁺ hydrocarbons;
C) cooling crude product stream B to obtain a hydrocarbon crude product stream C;
D) separating hydrocarbon crude product stream C into a propylene-comprising product of value stream, optionally an ethylene-comprising product of value stream, a butene-comprising product of value stream, at least one C₅-C₆ hydrocarbon-comprising recycle stream, and at least one by-product stream comprising C₆⁺ hydrocarbons;
E) recycling a portion of the C₂-C₄ olefins and at least a portion of the C₅-C₆ hydrocarbons as one or more hydrocarbon recycle streams R into step B);
F) obtaining a propylene-comprising product of value stream, an ethylene-comprising product of value stream, and optionally a product of value stream comprising butenes;
G) discharging the by-product stream comprising C₆+ hydrocarbons;
wherein stream A, based on methanol and ethanol, comprises <1% by weight or 30% to 50% by weight of ethanol, where, based on 100% by weight of the C₂-C₄ olefins obtained as products of value, 30% to 60% by weight of ethylene, 30% to 60% by weight of propylene and 0% to 30% by weight of butenes are obtained as products of value, and, based on the C₂-C₄ olefins present in crude product stream B, 0% to 40% of the ethylene, 40% to 90% of the propylene and 0% to 100% of the butenes are recycled into step B),
or stream A, based on methanol and ethanol, comprises 1% and 30% by weight of ethanol, where, based on 100% by weight of the C₂-C₄ olefins obtained as products of value, 0% to 20% by weight of ethylene, 70% to 100% by weight of propylene and 1% to 20% by weight of butenes are obtained as products of value, and, based on the C₂-C₄ olefins present in crude product stream B, 0% to 100% of the ethylene, 0% to 20% of the propylene and 40% to 100% of the butenes are recycled into step B).

In the process of the invention, based on 100% by weight of the C₂-C₄ olefins obtained as products of value, 30% to 60% by weight of ethylene, 30% to 60% by weight of propylene and 0% to 30% by weight of butenes are obtained as products of value, and, based on the C₂-C₄ olefins present in crude product stream B, 0% to 40% of the ethylene, 40% to 90% of the propylene and 0% to 100% of the butenes may be recycled into step B). The proportion of ethanol in the total amount of the alcohols ethanol and methanol fed to the process may be <1% by weight, especially 0% by weight. Alternatively, process economics may be improved when the proportion of ethanol in the alcohols fed to the process is 30% to 50% by weight.

Alternatively, the proportion of ethanol in the alcohols fed to the process overall may also be between 1% and 30% by weight, where, based on 100% by weight of the C₂-C₄ olefins obtained as products of value, 0% to 20% by weight of ethylene, 70% to 100% by weight of propylene and 1% to 20% by weight of butenes are obtained as products of value, and, based on the C₂-C₄ olefins present in crude product stream B, 0% to 100% of the ethylene, 0% to 20% of the propylene and 40% to 100% of the butenes are recycled into step B).

It has been found that it is possible by virtue of the altered recycling variants to achieve product compositions that would otherwise be possible only by virtue of very complex catalyst optimizations. Moreover, an addition of ethanol in the ranges of preference ascertained ensures a distinct reduction in the volume of recycle stream and by-products. This significantly reduces the energy demand of the process, since, for example, less mass has to be separated in a complex manner downstream of the reactor.

In a preferred embodiment, the process of the invention comprises the steps of:
A1) feeding a feed stream A comprising methanol, with or without ethanol, into a dimethyl ether fixed bed reactor and catalytically converting methanol to dimethyl ether and optionally ethanol to ethylene to obtain a product stream A1 comprising dimethyl ether, methanol and water vapor, with or without ethanol and ethylene;
A2) mixing at least a portion of product stream A1 with at least one hydrocarbon recycle stream R comprising C₂-C₆ hydrocarbons and a water vapor stream to obtain a second feed stream A2;
B1) heating the second feed stream A2 in one or more heat exchangers to a temperature in the range from 430 to 500° C. and feeding it into an olefin fixed bed reactor, where the heating may also precede the mixing of individual substreams to give the feed stream A2 in step A2;
B2) catalytically converting feed stream A2 at a temperature in the range from 430 520° C. to a crude product gas stream B comprising ethylene, propylene, butenes, further C₂-C₆ hydrocarbons, C₇⁺ hydrocarbons, methanol and water vapor;
C1) cooling crude product gas stream B in one or more heat exchangers to a temperature in the range from 170 to 220° C. by heat exchange with feed stream A2;
C2) further cooling crude product gas stream B to a temperature in the range from 30 to 60° C. by contacting with at least one water-containing quench circulation stream K, with condensation of water and methanol, to obtain a water- and methanol-depleted hydrocarbon crude product gas stream C;
D) separating hydrocarbon crude product gas stream C into a propylene-comprising product of value stream, optionally an ethylene-comprising product of value stream, a butene-comprising product of value stream, at least one recycle stream comprising C₅-C₆ hydrocarbons, and at least one by-product stream comprising C₆⁺ hydrocarbons.

In step A1), a feed stream A comprising methanol and ethanol is fed into a dimethyl ether fixed bed reactor and methanol is catalytically converted to dimethyl ether to obtain a product stream A1 comprising dimethyl ether, methanol, ethanol and water vapor.

If, based on 100% by weight of the C₂-C₄ olefins obtained as products of value, 30% to 60% by weight of ethylene, 30% to 60% by weight of propylene and 0% to 30% by weight of butenes are obtained as products of value, the proportion of ethanol in the total amount of the alcohols ethanol and methanol fed to the process may be <1% by weight. Alternatively, the proportion of ethanol may be increased to 30% to 50% by weight in order to achieve a more favorable operating window.

Alternatively, an advantageous proportion of ethanol in the total amount of the alcohols ethanol and methanol fed to the process is 1% to 30% by weight when, based on 100% by weight of the C₂-C₄ olefins obtained as products of value, 1% to 20% by weight of ethylene, 70% to 100% by weight of propylene and 0% to 20% by weight of butenes are obtained as products of value.

The dimethyl ether fixed bed reactor may be designed in various ways, depending on the exact composition of the feed gas stream. In general, the feed gas stream A comprising methanol, with or without ethanol, is heated up to a temperature of above 250° C. and fed into the fixed bed reactor. The catalyst used is generally gamma-alumina. The conversion temperature is between 25° and 450° C., and the pressure between 1 and 25 bar, for example 4 bar. The methanol conversion is generally 50% to 90%, preferably 65% to 85%, for example 75%.

In the case of small proportions of ethanol, stream A can be preheated to a temperature of 250 to 300° C., for example 275° C. Product gas stream A1 leaving the reactor is then at a temperature of generally 350 to 400° C., for example 370° C. This stream is then cooled to a temperature of generally 180 to 250° C. by heat exchange with stream A.

In the case of relatively high ethanol contents in stream A, stream A may also instead be heated up to temperatures of up to 450° C. and the reactor operated adiabatically. Alternatively, the reactor may be designed as a heated reactor, in which case stream A has to be heated merely to temperatures of up to 400° C.

In a step A2), at least a portion of said stream A1 is mixed with a hydrocarbon recycle stream R comprising C₂-C₆ hydrocarbons and a water vapor stream to obtain a feed gas stream A2. In general, this portion of stream A1 is at least 50% by weight and preferably up to 90% by weight. A further portion A1-2 of stream A1 of preferably at least 10% by weight can be fed directly to one or more trays of the olefin fixed bed reactor. This portion of stream A1 is generally cooled before being fed into the trays of the olefin fixed bed reactor, preferably to a temperature in the range from 30 to 60° C. This substream A1-2 is preferably fed into the reactor in liquid form.

The cooling by substream A1-2 can also be dispensed with if the olefin fixed bed reactor is operated and cooled isothermally. Isothermal operation can be effected, for example, in the manner described in WO 2017/102096 A1.

For instance, there may be heat transfer surfaces installed in the olefin fixed bed reactor that are operated, for example, with liquid salt or high-pressure steam as heat transfer medium. By virtue of the flow of the heat transfer medium through the heat transfer surfaces in the reactor, the heat of reaction that arises is removed from the reactor and hence this is operated isothermally. In order to establish the desired product distribution in product gas stream B, it is also possible here to feed a substream A1-2 to an intermediate stage of the olefin fixed bed reactor.

The at least one hydrocarbon recycle stream R that comes from the removal of the C₂-C₄ olefins generally comprises C₁-C₆ hydrocarbons. According to the proportions in which ethylene, propylene and butenes are obtained as products of value in step E), the at least one hydrocarbon recycle stream comprises C₂-C₄ hydrocarbons, generally in amounts of 50% to 90% by weight in total. The at least one hydrocarbon recycle stream prior to mixing is generally at a temperature in the range from 100 to 175° C., preferably in the range from 130 to 160° C.

The product stream A1 from the dimethyl ether fixed bed reactor is also mixed with a water vapor stream. The water vapor stream is generally at a temperature in the range from 100 to 200° C., preferably in the range from 100 to 150° C. The feed stream A2 thus obtained generally comprises 20% to 70% by weight, preferably 30% to 60% by weight, of water vapor. It generally further comprises 5% to 10% by weight of methanol, 0% to 15% by weight of ethanol, 10% to 20% by weight of dimethyl ether and 15% to 50% by weight of C₂-C₆ hydrocarbons.

In a step B1), the feed stream A2 is heated up in one or more heat exchangers to a temperature in the range from generally 430 to 500° C. and fed into an olefin fixed bed reactor. The heating may also take place prior to the mixing of individual substreams in step A2).

In general, feed stream A2 on feeding into the olefin fixed bed reactor is at a temperature in the range from 430 to 500° C., for example 470° C. Feed stream A2 can be heated to that temperature by heat exchange with the crude product gas stream B from the olefin fixed bed reactor, direct electrical heating, or heating via combustion of a separate fossil energy carrier.

This is followed in step B2) by catalytic conversion in the olefin fixed bed reactor to a product gas stream B comprising ethylene, propylene, butenes, further C₂-C₆ hydrocarbons, C₇⁺ hydrocarbons, methanol and water vapor. The conversion is generally effected on a zeolite catalyst, preferably over a catalyst based on a ZSM-5 zeolite. The reaction temperature is generally 430 to 500° C., preferably 460 to 480° C. The pressure is generally is 1.3 to 2.5 bar. The resultant crude product gas stream B2 is preferably of the following composition: 1% to 15% by weight of ethylene, 1% to 15% by weight of propylene, 35% to 80% by weight of water, 15% to 50% by weight of C₂-C₆ hydrocarbons, especially butenes, and saturated C₄ and C₅ hydrocarbons and C₆⁺ hydrocarbons, and also 0.01% to 1.5% by weight of methanol and dimethyl ether.

The olefin fixed bed reactor generally takes the form of a tray reactor. The number of trays is preferably 4 to 6. In one embodiment, a total of up to 50% by weight of the gas stream A is fed directly to one or more trays of the olefin fixed bed reactor, preferably all trays of the olefin fixed bed reactor. In a further embodiment, methanol is fed directly to one or more trays of the olefin fixed bed reactor, preferably all trays of the olefin fixed bed reactor.

The crude product gas stream B on exit from the reactor is at a temperature of generally 430 to 520° C., preferably 460 to 480° C.

In a preferred step C1), crude product gas stream B is cooled in one or more heat exchangers to a temperature in the range from 170 to 220° C. by heat exchange with feed gas stream A2. After this cooling step, the temperature of crude product gas stream B is generally 160 to 220° C., preferably 170 to 210° C., for example 190° C.

In a preferred step C2), crude product gas stream B is cooled further to a temperature in the range from 30 to 60° C. by contacting with one or more water-containing quench circulation streams, with condensation of water and methanol, to obtain a water- and methanol-depleted hydrocarbon crude product gas stream C. The hydrocarbon crude product gas stream C thus obtained comprises essentially ethylene, propylene, further C₂-C₆ hydrocarbons and C-+ hydrocarbons.

In a step D), one or more product streams comprising C₂-C₄ olefins are separated from the hydrocarbon crude product gas stream C, and the at least one recycle stream R comprising C₂-C₆ hydrocarbons is obtained. The (overall) recycle stream R may comprise or be formed from multiple individual recycle streams R1, R2, R3 etc. In general, the overall recycle stream R comprising C₂-C₆ hydrocarbons comprises essentially, i.e. to an extent of >95% by weight, C₂-C₆ hydrocarbons.

In general, step D) comprises steps D1) to D7):
D1) compressing hydrocarbon crude product gas stream C to obtain a liquid hydrocarbon stream D11 comprising propylene and C₄, C₅ and C₆⁺ hydrocarbons, and an ethane-, ethene- and propylene-comprising gaseous hydrocarbon stream D12;
D2) separating water from the liquid hydrocarbon stream D11 by phase separation to obtain a liquid hydrocarbon stream D21;
D3) separating a propylene-comprising stream D31 from the liquid hydrocarbon stream D21 to obtain a stream D32 comprising C₄, C₅ and C₈⁺ hydrocarbons; or separating a stream D31 comprising propylene and C₄ hydrocarbons to obtain a stream D32 comprising C₄, C₅ and C₆⁺ hydrocarbons;
D4) separating a by-product stream D41 comprising C₆⁺ hydrocarbons from the stream D32 comprising C₄, C₅ and C₆⁺ hydrocarbons to obtain a stream D42 comprising C₄, C₅ and C₆ hydrocarbons; stream D41 optionally comprises aromatic C₆ hydrocarbons, and stream D42 aliphatic C₆ hydrocarbons;
D5) separating a propylene-comprising stream D51 from the ethane-, ethene- and propylene-comprising gaseous hydrocarbon stream D12 to obtain an ethane- and ethene-comprising stream D52;
D6) separating a stream D61 comprising butenes from the stream D42 comprising C₄, C₅ and C₆ hydrocarbons to obtain a stream D62 comprising C₅ and C₆ hydrocarbons; and or separating a propylene-comprising stream D63 from stream D31 to obtain a stream D64 comprising butenes;
D7) obtaining at least one recycle stream R from one or more of the streams selected from stream D42 comprising C₄, C₅ and C₆ hydrocarbons, stream D62 comprising C₅ and C₆ hydrocarbons, the propylene-comprising stream D31, the propylene-comprising stream D51, the propylene-comprising stream D63, stream D61 comprising butenes, stream D64 comprising butenes, and the ethane- and ethene-comprising stream D52.

Steps D3), D4), D5) and D6) are conducted in standard distillation apparatuses. Useful distillation apparatuses in principle include the apparatuses known to the person skilled in the art for such separation tasks. As well as the actual column body with internals, the distillation column, as usual, also comprises a top condenser and a reboiler. The column body may have been equipped, for example, with structured packings, random packings or trays. The distillation apparatuses may be designed and operated by the common knowledge of the person skilled in the art.

In step E), a portion of the C₂-C₄ olefins and at least a portion of the C₅-C₆ hydrocarbons are recycled into step B) as one or more hydrocarbon recycle streams R.

In step F), a propylene-comprising product of value stream, an ethylene-comprising product of value stream, and optionally a product of value stream comprising butenes are obtained.

Propylene can be obtained as product of value from streams D31 or D63 and D51. Ethylene can be obtained as product of value from stream D52. Butenes can be obtained from stream D61 and/or D64.

The recycle stream(s) R may be obtained from one or more of the above-described streams D31, D42, D51, D52, D61, D62, D63 and D64.

Based on 100% by weight of the C₂-C₄ olefins obtained as products of value, 30% to 60% by weight of ethylene, 30% to 60% by weight of propylene and 0% to 30% by weight of butenes are obtained as products of value and, based on the C₂-C₄ olefins present in crude product stream B, 0% to 40% of the ethylene, 40% to 90% of the propylene and 0% to 100% of the butenes are recycled into step B), or, based on 100% by weight of the C₂-C₄ olefins obtained as products of value, 0% to 20% by weight of ethylene, 70% to 100% by weight of propylene and 1% to 20% by weight of butenes are obtained as products of value, and, based on the C₂-C₄ olefins present in crude product stream B, 0% to 100% of the ethylene, 0% to 20% of the propylene and 40% to 100% of the butenes are recycled into step B).

In step G), a by-product stream comprising C₆⁺ hydrocarbons is discharged from the process. This may be stream D41.

The invention is elucidated in detail by the examples below.

### EXAMPLES

The below ranges 1-4 of the composition of feed stream (ethanol content), crude product stream (ratios of C₂/C₃ olefins and C₄/C₃ olefins) and product of value obtained (proportions of C₂, C₃ and C₄ olefins) were simulated by calculation. Figures in weight ratios.

In order to ascertain the preferred ranges, catalytic experiments were conducted on a laboratory scale, on the basis of which the expected conversions and mass flow rates in an industrial process with a preliminary reactor, tray reactors and separation section were calculated. Optimization of this system led to the in ranges 1-4 that are specified in table 1. Table 2 shows the relevant flow rates for example points determined in ranges 1 and 2. Similar example values for ranges 3 and 4 are listed in table 3. The addition of ethanol in each case allows minimization of the relative recycle stream (Recycle) and the relative by-product stream, and hence improvement of the energy efficiency and mass efficiency of the process.

TABLE 1

| | Ratio of | | Proportion | Proportion | | |
|---|---|---|---|---|---|---|
| | C₂/C₃ | Ratio of | of | of | | |
| | olefins in | C₄/C₃ | recycled | recycled | Proportion | |
| | the | olefins | olefin in | olefin | of recycled | |
| | product | in the | the over- | in the | olefin in | |
| | of value | product of value | all C₂ olefin | overall C₃ olefin | the overall C₄ olefin | $\frac{EtOH}{EtOH + MeOH}$ |
| Range 1 | 0.8-1.2 | 0-0.7 | 0-0.4 | 0.4-0.9 | 0-1 | |
| Range 2 | 0.8-1.2 | 0-0.7 | 0-0.4 | 0.4-0.9 | 0-1 | 0.3-0.5 |
| Range 3 | 0-0.2 | 0.01-0.2 | 0-1 | 1 | 0-0.6 | |
| Range 4 | 0-0.2 | 0.01-0.2 | 0-1 | 1 | 0-0.6 | 0.01-0.3 |

The results of the simulations are collated in tables 2 and 3. All figures in weight ratios.

TABLE 2

| | 0 | | | 0.5 | | |
|---|---|---|---|---|---|---|
| $\frac{EtOH}{EtOH + MeOH}$ | Product | Recycle | By-products | Product | Recycle | By-products |
| Rel. mass flow | 1.0 | 3.73 | 1.88 | 1.0 | 0.63 | 0.53 |
| MeOH | | | | | | |
| EtOH | | | | | | |
| DME | | 0.01 | 0.01 | | | 0.02 |
| H₂O | | | | | | |
| H₂ | | | | | | |
| CO₂ | | | | | | |
| CH₄ | | | 0.02 | | | 0.01 |
| C₂H₄ | 0.39 | | | 0.41 | | |
| C₃H₆ | 0.41 | 0.39 | | 0.41 | 0.66 | |
| C₄H₈ | 0.20 | 0.12 | | 0.18 | 0.03 | |
| C₅H₁₀ | | 0.01 | 0.02 | | 0.02 | 0.02 |
| C₆-C₈ olefins | | 0.01 | 0.01 | | 0.02 | 0.03 |
| C₂H₆ | | | 0.08 | | | 0.04 |
| C₃H₈ | | 0.20 | 0.11 | | 0.11 | 0.13 |
| C₄H₁₀ | | 0.18 | 0.16 | | 0.02 | 0.19 |
| C₅H₁₂ | | 0.05 | 0.11 | | 0.07 | 0.08 |
| C₆-C₈ | | 0.03 | 0.06 | | 0.06 | 0.08 |
| paraffins | | | | | | |
| Aromatics | | | 0.42 | | | 0.39 |

TABLE 3

| | 0 | | | 0.21 | | |
|---|---|---|---|---|---|---|
| $\frac{EtOH}{EtOH + MeOH}$ | Product | Recycle | By-products | Product | Recycle | By-products |
| Rel. mass flow | 1.0 | 1.09 | 0.65 | 1.0 | 1.02 | 0.56 |
| MeOH | | | | | | |
| EtOH | | | | | | |
| DME | | 0.02 | 0.02 | | 0.01 | 0.02 |
| H₂O | | | | | | |
| H₂ | | | | | | |
| CO₂ | | | | | | |
| CH₄ | | | 0.01 | | | 0.01 |
| C₂H₄ | 0.02 | 0.15 | | 0.03 | 0.23 | |
| C₃H₆ | 0.85 | | | 0.86 | | |
| C₄H₈ | 0.13 | 0.18 | | 0.11 | 0.16 | |
| C₅H₁₀ | | 0.01 | 0.02 | | 0.01 | 0.02 |
| C₆-C₈ olefins | | 0.01 | 0.02 | | 0.01 | 0.03 |
| C₂H₆ | | 0.36 | 0.07 | | 0.34 | 0.07 |
| C₃H₈ | | | 0.10 | | | 0.12 |
| C₄H₁₀ | | 0.16 | 0.18 | | 0.14 | 0.17 |
| C₅H₁₂ | | 0.07 | 0.12 | | 0.06 | 0.10 |
| C₆-C₈ | | 0.04 | 0.06 | | 0.04 | 0.07 |
| paraffins | | | | | | |
| Aromatics | | | 0.39 | | | 0.39 |

1.-3. (canceled)

4. A process for preparing C₂-C₄ olefins from methanol and ethanol, comprising the steps of:
A) feeding a feed stream A comprising methanol, with or without ethanol, into a dimethyl ether fixed bed reactor and catalytically converting methanol to dimethyl ether to obtain a product stream A1 comprising dimethyl ether, methanol, ethanol and water vapor;
B) mixing stream A1 with at least one hydrocarbon recycle stream R comprising C₂-C₆ hydrocarbons and catalytically converting it in an olefin fixed bed reactor to a crude product stream B comprising C₂-C₄ olefins, C₅-C₆ hydrocarbon and C₇ hydrocarbons;
C) cooling crude product stream B to obtain a hydrocarbon crude product stream C;
D) separating hydrocarbon crude product stream C into a propylene-comprising product of value stream, optionally an ethylene-comprising product of value stream, at least one C₅-C₆ hydrocarbon-comprising recycle stream, and at least one by-product stream comprising C₆ hydrocarbons;
E) recycling a portion of the C₂-C₄ olefins and at least a portion of the C₅-C₆ hydrocarbons as one or more hydrocarbon recycle streams into step B);
F) obtaining a propylene-comprising product of value stream, an ethylene-comprising product of value stream, and optionally a product of value stream comprising butenes;
G) discharging the by-product stream comprising C₆ hydrocarbons;
wherein
stream A, based on methanol and ethanol, comprises <1% by weight or 30% to 50% by weight of ethanol, where, based on 100% by weight of the C₂-C₄ olefins obtained as products of value, 30% to 60% by weight of ethylene, 30% to 60% by weight of propylene and 0% to 30% by weight of butenes are obtained as products of value, and, based on the C₂-C₄ olefins present in crude product stream B, 0% to 40% of the ethylene, 40% to 90% of the propylene and 0% to 100% of the butenes are recycled into step B),
or stream A, based on methanol and ethanol, comprises 1% to 30% by weight of ethanol, where, based on 100% by weight of the C₂-C₄ olefins obtained as products of value, 0% to 20% by weight of ethylene, 70% to 100% by weight of propylene and 1% to 20% by weight of butenes are obtained as products of value, and, based on the C₂-C₄ olefins present in crude product stream B, 0% to 100% of the ethylene, 0% to 20% of the propylene and 40% to 100% of the butenes are recycled into step B).

5. The process according to claim 4, wherein steps A) to D) comprise the following steps A1), A2), B1), B2), C1), C2) and D):
A1) feeding a feed stream A comprising methanol and ethanol into a dimethyl ether fixed bed reactor and catalytically converting methanol to dimethyl ether to obtain a product stream A1 comprising dimethyl ether, methanol, ethanol and water vapor;
A2) mixing at least a portion of product stream A1 with at least one hydrocarbon recycle stream R comprising C₂-C₆ hydrocarbons and a water vapor stream to obtain a second feed stream A2;
B1) heating the second feed stream A2 in one or more heat exchangers to a temperature in the range from 430 to 500° C. and feeding it into an olefin fixed bed reactor, where the heating may also precede the mixing of individual substreams to give the feed stream A2 in step A2;
B2) catalytically converting feed stream A2 at a temperature in the range from 430 520° C. to a crude product gas stream B comprising ethylene, propylene, butenes, further C₂-C₆ hydrocarbons, C₇⁺ hydrocarbons, methanol and water vapor;
C1) cooling crude product gas stream B in one or more heat exchangers to a temperature in the range from 170 to 220° C. by heat exchange with feed stream A2;
C2) cooling crude product gas stream B to a temperature in the range from 30 to 60° C. by contacting with at least one water-containing quench circulation stream K, with condensation of water and methanol, to obtain a water- and methanol-depleted hydrocarbon crude product gas stream C2;
D) separating hydrocarbon crude product gas stream C into a propylene-comprising product of value stream, optionally an ethylene-comprising product of value stream, a butene-comprising product of value stream, at least one recycle stream comprising C₅-C₆ hydrocarbons, and at least one by-product stream comprising C₆⁺ hydrocarbons.

6. The process according to claim 4, wherein step D) comprises steps D1) to D7):
D1) compressing hydrocarbon crude product gas stream C to obtain a liquid hydrocarbon stream D11 comprising propylene and C₄, C₅ and C₆⁺ hydrocarbons, and an ethane-, ethene- and propylene-comprising gaseous hydrocarbon stream D12;
D2) separating water from the liquid hydrocarbon stream D11 by phase separation to obtain a liquid hydrocarbon stream D21;
D3) separating a propylene-comprising stream D31 from the liquid hydrocarbon stream D21 to obtain a stream D32 comprising C₄, C₅ and C₆⁺ hydrocarbons; or separating a stream D31 comprising propylene and C₄ hydrocarbons to obtain a stream D32 comprising C₄, C₅ and C₆⁺ hydrocarbons;
D4) separating a by-product stream D41 comprising C₆⁺ hydrocarbons from the stream D32 comprising C₄, C₅ and C₆⁺ hydrocarbons to obtain a stream D42 comprising C₄, C₅ and C₆ hydrocarbons; stream D41 optionally comprises aromatic C₆ hydrocarbons, and stream D42 aliphatic C₆ hydrocarbons;
D5) separating a propylene-comprising stream D51 from the ethane-, ethene- and propylene-comprising gaseous hydrocarbon stream D12 to obtain an ethane- and ethene-comprising stream D52;
D6) separating a stream D61 comprising butenes from the stream D42 comprising C₄, C₅ and C₆ hydrocarbons to obtain a stream D62 comprising C₅ and C₆ hydrocarbons; and or separating a propylene-comprising stream D63 from stream D31 to obtain a stream D64 comprising butenes;
D7) obtaining at least one recycle stream R from one or more of the streams selected from stream D42 comprising C₄, C₅ and C₆ hydrocarbons, stream D62 comprising C₅ and C₆ hydrocarbons, the propylene-comprising stream D31, the propylene-comprising stream D51, the propylene-comprising stream D63, stream D61 comprising butenes, stream D64 comprising butenes, and the ethane- and ethene-comprising stream D52.
